SEQUENCE LISTING (5 sequences)

SEQ ID NO: 1 - protein, 8 aa, Homo sapiens
MFSRPFRK

SEQ ID NO: 2 - protein, 11 aa, Homo sapiens
HGVVPLATYMR

SEQ ID NO: 3 - protein, 9 aa, Homo sapiens
  MISC_FEATURE (2)..(2): S-oxy-L-methionine
YXFSRPFRK

SEQ ID NO: 4 - protein, 14 aa, Homo sapiens
EPELLEPIPYEFMA

SEQ ID NO: 5 - protein, 14 aa, Homo sapiens
  MISC_FEATURE (13)..(13): S-oxy-L-methionine
EPELLEPIPYEFXA

(19) United States
(12) Patent Application Publication
(10) Pub. No.: US 2007/0004767 A1
(43) Pub. Date: Jan. 4, 2007

(54) Methods for treating neurofibromatosis 1

(76) Inventors: David H. Gutmann, St. Louis, MO (US); Jason D. Weber, St. Louis, MO (US)

Correspondence Address: Harness, Dickey & Pierce, P.L.C., Suite 400, 7700 Bonhomme Avenue, St. Louis, MO, 63105, US

(21) Appl. No.: 11/478,474

(22) Filed: Jun. 29, 2006

Related U.S. Application Data
(60) Provisional application No. 60/695,357, filed on Jun. 30, 2005.

(51) Int. Cl. A61K 31/4745

(52) U.S. Cl. 514/291

(57) ABSTRACT

This invention is directed generally to methods for treating neurofibromatosis 1 (NF1), and, more particularly, to methods for treating NF1 by administering rapamycin, a rapamycin analog, a rapamycin prodrug, or a salt of rapamycin or the analog or prodrug. This invention also is directed generally to compositions and kits for treating NF1, and more particularly, to compositions and kits for treating NF1 that comprise rapamycin, a rapamycin analog, a rapamycin prodrug, or a salt of rapamycin or the analog or prodrug.

### PRIORITY CLAIM TO RELATED PATENT APPLICATIONS

This patent application claims priority to U.S. Provisional Patent Application No. 60/695,357 (filed Jun. 30, 2005). The entire text of the '357 application is incorporated by reference into this application.

### STATEMENT AS TO RIGHTS TO INVENTIONS MADE UNDER FEDERALLY-SPONSORED RESEARCH AND DEVEOPMENT

This invention was made in part with financial support from the U.S. Government (i.e., Grant No. DAMD17-03-1-0215 from the Department of Defense). The U.S. Government has certain rights in this invention.

### FIELD OF THE INVENTION

This invention is directed generally to methods for treating neurofibromatosis 1 (NF1), and, more particularly, to methods for treating NF1 by administering rapamycin, a rapamycin analog, a rapamycin prodrug, or a salt of rapamycin or the analog or prodrug. This invention also is directed generally to compositions and kits for treating NF1, and more particularly, to compositions and kits for treating NF1 that comprise rapamycin, a rapamycin analog, a rapamycin prodrug, or a salt of rapamycin or the analog or prodrug.

### BACKGROUND OF THE INVENTION

NF1, also called von Recklinghausen neurofibromatosis or peripheral neurofibromatosis is believed to be the more common form of neurofibromatosis. It is estimated that it affects about one in 3000 individuals worldwide. Individuals with NF1 may develop hyperpigmented macules on the skin (“cafe-au-lait” spots), brain tumors (optic pathway glioma), benign and malignant peripheral nerve sheath tumors, freckling under the arms or in the groin area, hamartomas on the iris (Lisch nodules), and/or skeletal abnormalities including long bone dysplasia, curvature of the spine (scoliosis), or orbital dysplasia. Approximately half of the people with NF1 also have learning disabilities and/or attention deficit disorder.

Children with NF1 are prone to the development of World Health Organization (WHO) grade I astrocytomas typically located in the optic pathway, hypothalamus, or brainstem. These brain tumors can grow to cause early onset of puberty, loss of vision, and, in rare cases, death. In addition, some tumors will grow even after conventional chemotherapy. Adolescents and adults with NF1 typically develop benign Schwann cell tumors affecting the peripheral nervous system (neurofibromas). Neurofibromas are benign tumors, but a subset of these tumors (plexiform neurofibromas) may develop into a cancer (malignant peripheral nerve sheath tumor), which is frequently fatal. Adults with NF1 may also experience high blood pressure, headaches, chronic pain, and/or itching of the skin.

It has been shown that the NF1 gene encodes a protein, neurofibromin 1 (neurofibromin), which functions as a RAS-GTPase activating protein (RAS-GAP). In this regard, loss of neurofibromin in NF1-associated tumors is associated with high levels of RAS activation. The RAS-GAP function of neurofibromin provided the impetus for the use of anti-RAS therapies for NF1-associated tumors. RAS activation requires isoprenylation. Isoprenylation has been shown to be blocked by farnesyl transferase inhibitors in vitro and in vivo. While in preclinical studies treatment of both NF1-deficient human and Nf1−/− mouse cells with farnesyl transferase inhibitors resulted in reduction in cell proliferation, the use of the farnesyl transferase inhibitors in patients with NF1 reportedly has demonstrated little effect on tumor growth. See Packer B J et al., Plexiform Neurofibromas in NF1: Toward Biologic-Based Therapy, NEUROLOGY 58:1461-1470 (2000).

Applicants are unaware of any cure for NF1. Thus, there continues to be a need for methods of treatments for NF1, which provide partial or complete relief. This invention provides methods of treatment that generally address such a need.

### SUMMARY OF THE INVENTION

This invention is directed generally to methods for treating NF1, and, more particularly, to methods for treating NF1 by administering rapamycin, a rapamycin analog, a rapamycin prodrug, or a salt of rapamycin or the analog or prodrug.

Briefly, therefore, this invention is directed, in part, to a method for treating NF1 in an animal, wherein the method comprises administering to the animal an amount of rapamycin or a pharmaceutically-acceptable salt thereof.

This invention also is directed, in part, to a method for treating NF1 in an animal, wherein the method comprises administering to the animal an amount of a rapamycin analog, a rapamycin prodrug, or a pharmaceutically-acceptable salt of the analog or prodrug.

This invention also is directed generally to a method for determining if an animal neurofibromatosis 1-associated tumor is likely to respond to treatment with rapamycin, a rapamycin analog, a rapamycin prodrug, or a pharmaceutically-acceptable salt of rapamycin or the rapamycin analog or prodrug. The method comprises examining the level of phosphorylation of ribosomal S6 protein in a sample obtained from the tumor.

This invention also is directed generally to a use of rapamycin, a rapamycin analog, a rapamycin prodrug, or a pharmaceutically-acceptable salt of rapamycin or the analog or prodrug to prepare a medicament for treating NF1.

This invention also is directed generally to a composition and kit for treating NF1, and more particularly, to a composition and kit for treating NF1 that comprise an amount of rapamycin, a rapamycin analog, a rapamycin prodrug, or a salt of rapamycin or the analog or prodrug.

Further benefits of Applicants' invention will be apparent to one skilled in the art from reading this patent.

### BRIEF DESCRIPTION OF THE DRAWINGS

FIG. 1A shows (1) results from proteomic analysis and MALDI-TOF mass spectrometry of Nf1+/+ and Nf1−/− astrocyte protein lysates; and (2) peptide sequences for significant peptide matches: MFSRPFRK (SEQ ID NO:1), HGVVPLATYMR (SEQ ID NO:2), YM_{OX}FSRPFRK (SEQ ID NO:3), EPELLEPIPYEFMA (SEQ ID NO:4), and EPELLEPIPYEFM_{OX}A (SEQ ID NO:5).

FIG. 1B shows close-up views of two-dimensional gels stained with SYPRO-Ruby, and depicting differential expression of proteins extracted from Nf1+/+ and Nf1−/− astrocytes.

FIG. 1C shows immunoblots of Nf1+/+ and Nf1−/− astrocyte protein lysates with antibodies recognizing α-tubulin, fibrillarin, nucleophosmin, nucleolin, ribosomal protein L7, and cyclin D1.

FIG. 2A shows increased protein synthesis as measured by ³⁵S-methionine incorporation in Nf1−/− astrocytes as a function of time of ³⁵S-methionine exposure in the presence (+Rap) or absence of rapamycin. The mean and standard deviation is shown for each time point. Asterisks denote a statistically significant difference (P<0.001).

FIG. 2B shows (1) adenoviral Cre infection of Nf1^{flox/flox} astrocytes compared to control Nf1^{flox/flox} astrocytes infected with adenoviral LacZ; and (2) increased levels of ribosomal S6 protein activation in serum-deprived Nf1−/− astrocytes (Nf1^{flox/flox};AdCre) and Nf1+/+ astrocytes (Nf1^{flox/flox};AdLacZ). α-tubulin expression is included as a loading control (lower panel).

FIG. 2C shows that ribosomal S6 protein hyperactivation in Nf1−/− astrocytes is dependent on phosphatidylinositol 3-kinase (PI3K) and mammalian target of rapamycin (mTOR).

FIG. 3A shows expression of KT3 epitope-tagged NF1GRD in Nf1−/− astrocytes transduced with MSCV-NF1GRD or MSCV-Pac (vector control).

FIG. 3B shows that ribosomal S6 protein phosphorylation is increased in Nf1+/+ astrocytes, and that the increased phosphorylation is inhibited by transduction of the NF1GRD (MSCV-NF1GRD). α-tubulin expression is included as a loading control (lower panel).

FIG. 4A shows that oncogenic K-RAS^{G12D} expression in astrocytes (K-RAS^{GFAP}) results in increased phosphorylation of ribosomal S6 protein, compared to control (wild-type) astrocytes.

FIG. 4B shows expression of T7 epitope-tagged dominant inhibitory K-RAS^{N17} (MSCV-K-RAS^{N17}-GFP) in Nf1−/− astrocytes after viral transduction. Fluorescence microscopy demonstrates 100% transduction (GFP).

FIG. 4C shows that the increased ribosomal S6 protein phosphorylation in Nf1−/− astrocytes is inhibited by expression of a dominant inhibitory K-RAS protein (K-RAS^{N17}). α-tubulin expression is included as a loading control (lower panel).

FIG. 5 shows an increase in ribosomal S6 protein phosphorylation in (1) two models of mouse Nf1 optic pathway glioma: Nf1 heterozygous mice with astrocyte-specific Nf1 inactivation (B), or astrocyte-specific oncogenic K-RAS expression (D) (control mouse optic nerve sections are in panels A and C, respectively); (2) two human NF1-associated pilocytic astrocytomas (E, F) (phosphorylated ribosomal S6 protein in a focus of neoplastic cells with enlarged atypical nuclei in one case is shown in the inset (F)).

FIG. 6 shows dose-dependent inhibition of ribosomal S6 protein phosphorylation and cell proliferation (growth) in Nf1−/− astrocytes by rapamycin (there is little or no effect of rapamycin treatment on control astrocytes). Asterisks denote a statistically significant difference (P<0.001).

### DETAILED DESCRIPTION

This detailed description is intended only to acquaint others skilled in the art with Applicants' invention, its principles, and its practical application so that others skilled in the art may adapt and apply the invention in its numerous forms, as they may be best suited to the requirements of a particular use. This description and its specific examples are intended for purposes of illustration only. This invention, therefore, is not limited to the embodiments described in this application, and may be variously modified.

This invention is directed, in part, to a method for treating NF1. “Treating” means ameliorating, suppressing, eradicating, preventing, reducing the risk of, and/or delaying the onset of the disease being treated. The method of treatment is particularly suitable for use with humans, but may be used with other animals, particularly mammals, such as non-human primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

In some embodiments, the animal has a benign tumor. In some such embodiments, the tumor comprises a neurofibroma. In other such embodiments, the tumor comprises an astrocytoma. In further such embodiments, the tumor comprises a plexiform neurofibroma.

In some embodiments, the animal has a malignant tumor. In some such embodiments, the tumor is a malignant peripheral nerve sheath tumor (MPNST or sarcoma).

In some embodiments, the animal has a skeletal abnormality. In some such embodiments, the skeletal abnormality comprises orbital dysplasia (thinning of the bones of the orbit). In other such embodiments, the skeletal abnormality comprises dysplasia of the long bones of the extremities (tibia, fibula, ulna, or radius). In further such embodiments, the skeletal abnormality comprises curvature of the spine (scoliosis).

In some embodiments, the animal has a learning disability.

In some embodiments, the animal has attention deficit disorder.

In some embodiments, the animal has hamartomas on the iris.

In some embodiments, the method for treating NF1 comprises administering to an animal in need of treatment a therapeutically-effective amount of rapamycin or a salt thereof. A “therapeutically-effective amount” or “effective amount” means an amount that will achieve the goal of treating the targeted condition.

Rapamycin (also known as sirolimus) is a naturally-occurring and commercially available (Sigma, St. Louis, Mo.) macrolide antibiotic corresponding in structure to formula I:

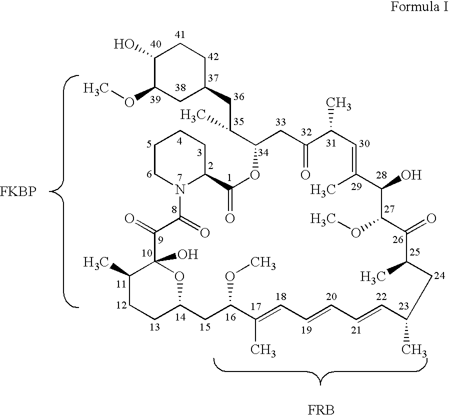

Rapamycin binds with high affinity to the 12 kDa FK506 binding protein (FKBP12), as well as to a 100-amino acid domain (E2015 to Q2114) of the mammalian target of rapamycin (mTOR) known as the FKBP-rapamycin binding domain (FRB). The portions of rapamycin that bind to FKBP12 and FRB are shown in formula I.

In some embodiments, the method for treating NF1 comprises administering a rapamycin analog or a salt thereof. A “rapamycin analog” means a compound having a structure similar to that of rapamycin, but differing from it in respect to one or more components. Thus, a rapamycin analog encompasses a rapamycin isomer such as, for example, 28-epirapamycin, as well as 28-epirapamycin analogs.

It should also be noted that throughout the scientific and patent literature, different methods of numbering the atoms in the rapamycin structure have been used. Thus, rapamycin analogs can have different names depending on the numbering method used. This patent uses the numbering provided in Formula I above. One skilled in the art can easily correlate the names of the rapamycin analogs that are named using a different numbering method.

In some embodiments, the method for treating NF1 comprises administering a rapamycin prodrug or a salt thereof. A “prodrug” means a compound that transforms into rapamycin upon administration. For example, CCI-779 is a rapamycin prodrug because upon administration it transforms to yield rapamycin.

In some embodiments, the method for treating NF1 comprises administering a hydroxyester rapamycin analog selected from the rapamycin analogs discussed in U.S. Pat. No. 5,362,718. In some such embodiments, for example, the rapamycin analog is rapamycin 40-O-ester with 3-hydroxy-2-hydroxymethyl-2-methyl-propionic acid (also known as CCI-779 or temsirolimus):

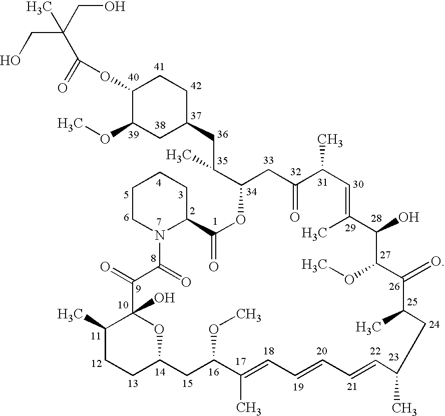

In some embodiments, the method for treating NF1 comprises administering an O-alkylated rapamycin analog selected from the rapamycin analogs discussed in U.S. Pat. No. 6,440,990. In some such embodiments, for example, the rapamycin analog is 40-O-(2-hydroxy)ethyl rapamycin (also known as RAD001 or everolimus):

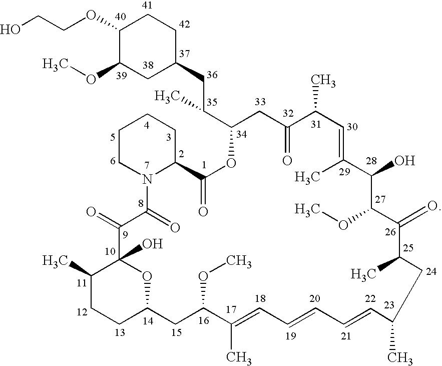

In some embodiments, the method for treating NF1 comprises administering a phosphorus-containing rapamycin analog selected from the rapamycin analogs discussed in International Publication No. WO 03/064383 A2. In some such embodiments, for example, the rapamycin analog is a rapamycin 40-O-ester with dimethyl-phosphinic acid (also known as AP23573):

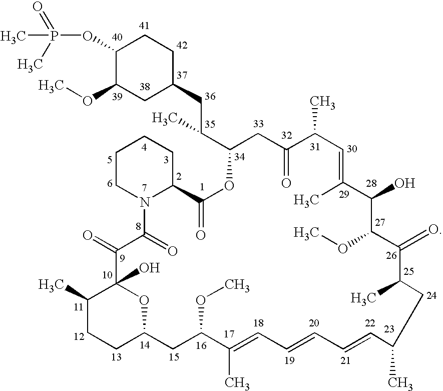

In some embodiments, the method of this invention comprises a combination therapy, wherein rapamycin, a rapamycin analog, a rapamycin prodrug, or a salt of rapamycin or the analog or prodrug is co-administered with a second (or even a third, fourth, etc.) compound, such as, for example, an anti-tumor or an anti-inflammatory compound. An “anti-tumor compound” means a compound that treats tumor(s). An “anti-inflammatory compound” means a compound that treats inflammation. In these embodiments, rapamycin (or an analog, prodrug, or salt) and the second compound (or a salt thereof) may be administered in a substantially simultaneous manner (e.g., within about 5 minutes of each other), in a sequential manner, or both. It is contemplated that such combination therapies may include administering one compound multiple times between the administration of the other compound. The time period between the administration of each compound may range from a few seconds (or less) to several hours or days, and will depend on, for example, the properties of each composition and active ingredient (e.g., potency, solubility, bioavailability, half-life, and kinetic profile), as well as the condition of the patient.

In some embodiments, rapamycin, a rapamycin analog, a rapamycin prodrug, or a salt of rapamycin or the analog or prodrug is co-administered with an anti-tumor compound or a pharmaceutically-acceptable salt thereof. Suitable anti-tumor compounds include, for example, alkylating agents (e.g., carboplatin, temozolomide, 1,3 bis(2-chloroethyl)-1-nitrosourea (BCNU)), anti-angiogenic agents (e.g., irinotecan, Cox-2 inhibitors), receptor tyrosine kinase inhibitors (e.g., imatinib (Glivec)), and RAS/RAF inhibitors (e.g., farnesyltransferase inhibitors, sorafenib (BAY43-9000)). In some such embodiments, rapamycin, a rapamycin analog, a rapamycin prodrug, or a salt of rapamycin or the analog or prodrug is co-administered with an alkylating anti-tumor compound or a pharmaceutically-acceptable salt thereof. In some such embodiments, the alkylating anti-tumor compound comprises carboplatin.

Rapamycin and rapamycin analogs and prodrugs can be used in the form of salts derived from inorganic or organic acids. Depending on the particular compound, a salt of the compound may be advantageous due to one or more of the salt's physical properties, such as enhanced pharmaceutical stability in differing temperatures and humidities, or a desirable solubility in water or oil.

The salt preferably is pharmaceutically acceptable. Pharmaceutically-acceptable salts include salts commonly used to form alkali metal salts and to form addition salts of free acids or free bases. In general, these salts typically may be prepared by conventional means with rapamycin or a rapamycin analog or prodrug by reacting, for example, the appropriate acid or base with rapamycin or a rapamycin analog or prodrug.

Pharmaceutically-acceptable acid addition salts of rapamycin or rapamycin analogs or prodrugs may often be prepared from an inorganic or organic acid. Examples of often suitable inorganic acids include hydrochloric, hydrobromic, hydroiodic, nitric, carbonic, sulfuric, and phosphoric acid. Suitable organic acids generally include, for example, aliphatic, cycloaliphatic, aromatic, araliphatic, heterocyclic, carboxylic, and sulfonic classes of organic acids. Specific examples of often suitable organic acids include acetate, trifluoroacetate, formate, propionate, succinate, glycolate, gluconate, digluconate, lactate, malate, tartaric acid, citrate, ascorbate, glucuronate, maleate, fumarate, pyruvate, aspartate, glutamate, benzoate, anthranilic acid, mesylate, stearate, salicylate, p-hydroxybenzoate, phenylacetate, mandelate, embonate (pamoate), ethanesulfonate, benzenesulfonate, pantothenate, 2-hydroxyethanesulfonate, sulfanilate, cyclohexylaminosulfonate, algenic acid, beta-hydroxybutyric acid, galactarate, galacturonate, adipate, alginate, bisulfate, butyrate, camphorate, camphorsulfonate, cyclopentanepropionate, dodecylsulfate, glycoheptanoate, glycerophosphate, heptanoate, hexanoate, nicotinate, 2-naphthalesulfonate, oxalate, palmoate, pectinate, 3-phenylpropionate, picrate, pivalate, thiocyanate, tosylate, and undecanoate.

Pharmaceutically-acceptable base addition salts of rapamycin and rapamycin analogs and prodrugs include, for example, metallic salts and organic salts. Preferred metallic salts include alkali metal (group Ia) salts, alkaline earth metal (group IIa) salts, and other physiologically acceptable metal salts. Such salts may be made from aluminum, calcium, lithium, magnesium, potassium, sodium, and zinc. Preferred organic salts can be made from amines, such as tromethamine, diethylamine, N,N′-dibenzylethylenediamine, chloroprocaine, choline, diethanolamine, ethylenediamine, meglumine (N-methylglucamine), and procaine. Basic nitrogen-containing groups can be quaternized with agents such as lower alkyl (C₁-C₆) halides (e.g., methyl, ethyl, propyl, and butyl chlorides, bromides, and iodides), dialkyl sulfates (e.g., dimethyl, diethyl, dibutyl, and diamyl sulfates), long chain halides (e.g., decyl, lauryl, myristyl, and stearyl chlorides, bromides, and iodides), aralkyl halides (e.g., benzyl and phenethyl bromides), and others.

This invention also is directed to a use of rapamycin, a rapamycin analog, a rapamycin prodrug, or a pharmaceutically-acceptable salt of rapamycin or the analog or prodrug to prepare a medicament for treating NF1.

This invention further is directed to a pharmaceutical composition comprising rapamycin, a rapamycin analog, a rapamycin prodrug, or a salt of rapamycin or the analog or prodrug, and to a method for making a pharmaceutical composition comprising rapamycin, a rapamycin analog, a rapamycin prodrug, or a salt of rapamycin or the analog or prodrug.

The preferred composition (medicament) depends on the method of administration, and typically comprises one or more conventional pharmaceutically acceptable carriers, adjuvants, and/or vehicles (together referred to as “excipients”). Formulation of drugs is generally discussed in, for example, Hoover, J., Remington's Pharmaceutical Sciences (Mack Publishing Co., 1975); and Allen, Jr., L V et al., Ansel's Pharmaceutical Dosage Forms and Drug Delivery Systems (Lippincott Williams & Wilkins, 2005).

Solid dosage forms for oral administration include, for example, capsules, tablets, pills, powders, and granules. In such solid dosage forms, the compounds or salts are ordinarily combined with one or more excipients. If administered per os, the compounds or salts can be mixed with lactose, sucrose, starch powder, cellulose esters of alkanoic acids, cellulose alkyl esters, talc, stearic acid, magnesium stearate, magnesium oxide, sodium and calcium salts of phosphoric and sulfuric acids, gelatin, acacia gum, sodium alginate, polyvinylpyrrolidone, and/or polyvinyl alcohol, and then tableted or encapsulated for convenient administration. Such capsules or tablets can contain a controlled-release formulation, as can be provided in a dispersion of the compound or salt in hydroxypropylmethyl cellulose. In the case of capsules, tablets, and pills, the dosage forms also can comprise buffering agents, such as sodium citrate, or magnesium or calcium carbonate or bicarbonate. Tablets and pills additionally can be prepared with enteric coatings.

Liquid dosage forms for oral administration include, for example, pharmaceutically-acceptable emulsions (including both oil-in-water and water-in-oil emulsions), solutions (including both aqueous and non-aqueous solutions), suspensions (including both aqueous and non-aqueous suspensions), syrups, and elixirs containing inert diluents commonly used in the art (e.g., water). Such compositions also can comprise, for example, wetting, emulsifying, suspending, flavoring (e.g., sweetening), and/or perfuming agents.

Parenteral administration includes subcutaneous injections, intravenous injections, intramuscular injections, intrasternal injections, and infusion. Injectable preparations (e.g., sterile injectable aqueous or oleaginous suspensions) can be formulated according to the known art using suitable dispersing, wetting agents, and/or suspending agents. Acceptable vehicles and solvents include, for example, water, 1,3-butanediol, Ringer's solution, isotonic sodium chloride solution, bland fixed oils (e.g., synthetic mono- or diglycerides), fatty acids (e.g., oleic acid), dimethyl acetamide, surfactants (e.g., ionic and non-ionic detergents), and/or polyethylene glycols.

Formulations for parenteral administration may, for example, be prepared from sterile powders or granules having one or more of the excipients mentioned for use in the formulations for oral administration. Rapamycin, a rapamycin analog, a rapamycin prodrug, or a salt of rapamycin or the analog or prodrug can be dissolved in water, polyethylene glycol, propylene glycol, ethanol, corn oil, cottonseed oil, peanut oil, sesame oil, benzyl alcohol, sodium chloride, and/or various buffers. The pH may be adjusted, if necessary, with a suitable acid, base, or buffer.

Suppositories for rectal administration can be prepared by, for example, mixing rapamycin, a rapamycin analog, a rapamycin prodrug, or a salt of rapamycin or the analog or prodrug with a suitable nonirritating excipient that is solid at ordinary temperatures, but liquid at the rectal temperature, and will therefore melt in the rectum to release the drug. Suitable excipients include, for example, cocoa butter; synthetic mono-, di-, or triglycerides; fatty acids; and/or polyethylene glycols.

Topical administration includes the use of transdermal administration, such as transdermal patches or iontophoresis devices.

Other excipients and modes of administration known in the pharmaceutical art also may be used.

The preferred total daily dose of rapamycin, rapamycin analog, rapamycin prodrug or salt of rapamycin or the analog or prodrug (administered in single or divided doses) is typically from about 0.001 to about 100 mg/kg, more preferably from about 0.001 to about 30 mg/kg, and even more preferably from about 0.01 to about 10 mg/kg (i.e., mg of rapamycin, rapamycin analog, rapamycin prodrug or salt of rapamycin or the analog or prodrug per kg body weight). Dosage unit compositions can contain such amounts or submultiples thereof to make up the daily dose. In many instances, the administration of the compound or salt will be repeated a plurality of times. Multiple doses per day typically may be used to increase the total daily dose, if desired.

Factors affecting the preferred dosage regimen include the type, age, weight, sex, diet, and condition of the patient; the severity of the pathological condition; the severity of the pathological condition; the route of administration; pharmacological considerations, such as the activity, efficacy, pharmacokinetic, and toxicology profiles of the particular compound or salt used; whether a drug delivery system is utilized; and whether the compound or salt is administered as part of a drug combination. Thus, the dosage regimen actually employed can vary widely, and therefore, can derive from the preferred dosage regimen set forth above.

This invention also is directed to a kit for treating NF1 in an animal. The kit comprises a dosage form comprising an amount of rapamycin, a rapamycin analog, a rapamycin prodrug, or a salt of rapamycin or the analog or prodrug.

In some embodiments, the kit comprises a dosage form comprising a therapeutically-effective amount of rapamycin, a rapamycin analog, a rapamycin prodrug, or a pharmaceutically-acceptable salt of rapamycin or the analog or prodrug.

In some embodiments, the kit further comprises a second dosage form comprising a second (or third, fourth, etc.) compound, for example an anti-cancer or anti-inflammatory compound or a pharmaceutically-acceptable salt thereof. In some such embodiments, the total amount of rapamycin, or a rapamycin analog or prodrug (or a pharmaceutically-acceptable salt thereof) and the second compound is a therapeutically-effective amount. In other such embodiments, the kit further comprises a second dosage form comprising carboplatin or a pharmaceutically-acceptable salt thereof. In some such embodiments, the amount of rapamycin, or a rapamycin analog or prodrug (or a pharmaceutically-acceptable salt thereof) and the amount of carboplatin (or a salt thereof) together comprise a therapeutically-effective amount.

Many drugs have undesirable side effects. In addition, patients treated with the same drug for the same disease do not always respond in the same manner. Thus, it is useful to have an assay that can predict if an animal in need of a particular treatment is likely to respond to that treatment. Thus, this invention also is directed to a method for determining if an animal NF1-associated tumor is likely to respond to treatment with rapamycin, a rapamycin analog, a rapamycin prodrug, or a pharmaceutically-acceptable salt of rapamycin or the analog or prodrug. The method comprises examining the level of phosphorylation of ribosomal S6 protein in a sample obtained from the tumor. An increased level of phosphorylation of ribosomal S6 protein in the tumor sample indicates that the tumor is likely to respond to treatment with rapamycin or a rapamycin analog.

In some embodiments, the level of phosphorylation of ribosomal S6 protein in the tumor sample is compared to the level of phosphorylation of ribosomal S6 protein in a control sample. In some such embodiments, a control sample is obtained from an area unaffected by a tumor.

In some embodiments, the NF1-associated tumor being examined is a human tumor.

In some embodiments, the NF1-associated tumor being examined is a mammalian tumor.

In some embodiments, the NF1-associated tumor being examined is a benign tumor. In some such embodiments, the tumor is a neuroblastoma. In other such embodiments, the tumor is an astrocytoma.

In some embodiments, the tumor being examined is a malignant tumor. In some such embodiments, the tumor is a malignant peripheral nerve sheath tumor.

In some embodiments, the sample obtained from the NF1-associated tumor is prepared as a paraffin-embedded biopsy.

In some embodiments, the sample obtained from the NF1-associated tumor is prepared as a snap-frozen tissue.

In some embodiments, examining the level of phosphorylation of ribosomal S6 protein comprises treating the sample obtained from the tumor with an antibody that recognizes one or more phosphorylated serine residues of ribosomal S6 protein.

In some embodiments, the method for determining if an animal NF1-associated tumor is likely to respond to treatment with rapamycin, a rapamycin analog, a rapamycin prodrug, or a pharmaceutically-acceptable salt of rapamycin or the analog or prodrug is performed as described in Example 6.

### EXAMPLES

The following examples are merely illustrative, and not limiting to this disclosure in any way.

### Example 1

### Materials and Methods

This example describes the materials and methods used in Examples 2-7 below.

### A. Mice

GFAP-Cre-IRES-LacZ transgenic (GFAPCre) mice were generated as described in Bajenaru M L et al., Astrocyte-Specific Inactivation of the Neurofibromatosis 1 Gene (NF1) is Insufficient for Astrocytoma Formation, MOL. CELL. BIOL. 22:5100-5113 (2002). Lox-stop-lox (LSL)-K-RAS^{G12D} mice were provided by Dr. Tyler Jacks from the Massachusetts Institute of Technology, Boston, Mass., and can be generated as described in Jackson E L, et al., Analysis of Lung Tumor Initiation and Progression Using Conditional Expression of Oncogenic K-ras, GENES DEV. 15:3243-3248 (2001). Lox-stop-lox (LSL)-K-RAS^{G12D} mice were crossed with GFAPCre mice to obtain (LSL)-K-RAS^{G12D}; GFAPCre mice. Nf1^{flox/flox} mice were provided by Dr. Luis Parada from the University of Texas Southwestern Medical Center, Dallas, Tex., and are available from the Mouse Models for Human Cancers Consortium E-Mice Repository (http://emice.nci.nih.gov). Mice were used in accordance with established animal studies protocols at Washington University, St. Louis, Mo.

### B. Primary Astrocyte Cultures

Murine neocortical astroglial cultures, containing >95% GFAP positive cells (astrocytes), were generated from postnatal day 2 Nf1^{flox/flox} and (LSL)-K-RAS^{G12}D; GFAPCre transgenic pups as previously described. See Bajenaru M L, et al., MOL. CELL BIOL. 22:5100-5113 (2002). To inactivate Nf1 expression, Nf1^{flox/flox} astrocytes were treated with Ad5Cre (University of Iowa Gene Transfer Vector core, Iowa City) or Ad5-lacZ (control), and neurofibromin loss was confirmed by Western blot. Astrocyte proliferation assays were performed by ³[H]-thymidine incorporation.

### C. Proteomic Analysis

Astrocytes were lysed in rehydration/sampling buffer (BioRad), and protein concentrations determined using the DC assay (BioRad). 200 μg of proteins and 12.5 μL of 200 mM tributylphosphine (BioRad) were mixed, and incubated for 20 min at room temperature. Sample loading for the first dimension was performed by passive in-gel rehydration. IPG strips (BioRad) were focused according to standard protocol, and stopped at 30,000 Vh. After the first dimension, the strips were equilibrated for 20 min in buffer containing 6M urea, 0.375M Tris, pH8.8, 20% glycerol, 2% TWEEN-20, 0.2% SDS, 1 mM EDTA with 2% DTT, and washed for 10 min in the same buffer without DTT, and then equilibrated for 20 min in the same buffer with 5% iodoacetamide instead of DTT. Prior to the second dimension, the strips were rinsed in 1×SDS PAGE running buffer, and placed on the top of 10% SDS-PAGE with 4% stacking gel. Separated proteins were stained with SYPRO-Ruby (BioRad) according to manufacturer's protocol. Gel images were analyzed using PDQuest software (BioRad). Spots of interest were excised and processed for trypsin digestion. Tryptic peptides were calibrated with Sequazyme peptide mass standards kit (PE Biosystem), and analyzed by MALDI-TOF mass spectrometry (Voyager DE Pro, Applied Biosystems). Identification of proteins was performed using MS-Fit software (http://prospector.ucsf.edu/ucsfhtml4.0/msfit.htm).

### D. Western Immunoblot Analysis

Western blot analysis was performed as described previously in, for example, Dasgupta B et al., The Neurofibromatosis 1 Gene Product Neurofibromin Regulates Pituitary Adenylate Cyclase-Activating Polypeptide-Mediated Signaling in Astrocytes, J. NEUROSCI. 23:8949-8954 (2003). The following antibodies were used: anti-NF1GRP, nucleolin, and cyclin D1 (Santa Cruz Biotechnology, Santa Cruz, Calif.), phospho-S6 (Serine-240/244; Cell Signaling Technology, Beverley, Mass.), α-tubulin and fibrillarin (Sigma, St. Louis, Mo.), anti-T7 (Novagen, Madison, Wis.), anti-KT3 (Babco, Richmond, Calif.), NPM (Zymed, San Francisco, Calif.), and L7 (Novus Biologicals, Littleton, Colo.).

### E. Protein Translation

Astrocytes were incubated with dialyzed serum (5%) in the absence of methionine for 30 min. ³⁵S-methionine was added to the culture medium and astrocytes were incubated at 37° C. for 5, 10, 15, 30, and 45 min in the presence or absence of rapamycin. At the end of each incubation period, astrocytes were harvested in RIPA buffer (50 mM Tris pH7.4, 150 mM NaCl, 1% Triton X-100, 0.1% SDS, 0.5% deoxycholic acid). Proteins were precipitated with trichloroacetic acid. ³⁵S-methionine-labeled proteins were detected by scintillation counting.

### F. Retroviral Transduction of Astrocytes

Retroviral transduction was performed as described in Hiatt K K, et al., Neurofibromin GTPase-Activating Protein-Related Domains Restore Normal Growth in NF1−/− Cells, J. BIOL. CHEM. 276:7240-7245 (2001). Expression of NF1GRD was confirmed by Western blot. Dominant negative K-RAS (K-RAS^{N17}) was generated by site directed mutagenesis as described in Walsh A B & Bar-Sagi D, Differential Activation of the Rac Pathway by Ha-Ras and K-Ras, J. BIOL. CHEM. 276:15609-15615 (2001); and Chen Z, et al., The C-Terminal Polylysine Region and Methylation of K-ras are Critical for the Interaction Between K-ras and Microtubules, J. BIOL. CHEM. 275:41251-41257 (2000). Transduction of primary astrocytes was performed using MSCV-K-RAS^{N17}-IRES-GFP and MSCV-IRES-GFP (control) as described in Uhlmann E J, et al., Loss of Tuberous Sclerosis Complex 1 (Tsc1) Expression Results in Increased Rheb/S6K Pathway Signaling Important for Astrocyte Cell Size Regulation, GLIA 47:180-188 (2004).

### G. Immunohistological Analysis

Immunohistochemical analysis was performed on paraffin-embedded mouse optic nerve sections and human pilocytic astrocytomas with rabbit anti-phospho S6 antibodies as described in Bajenaru M L, et al., MOL. CELL BIOL. 22:5100-5113 (2002). Human tumors were used in accordance with active human studies protocols at Washington University, St. Louis, Mo.

### Example 2

### Proteomic Analysis Uncovers Hyperactivation of the mRNA Translation Machinery in Nf1-Deficient Astrocytes

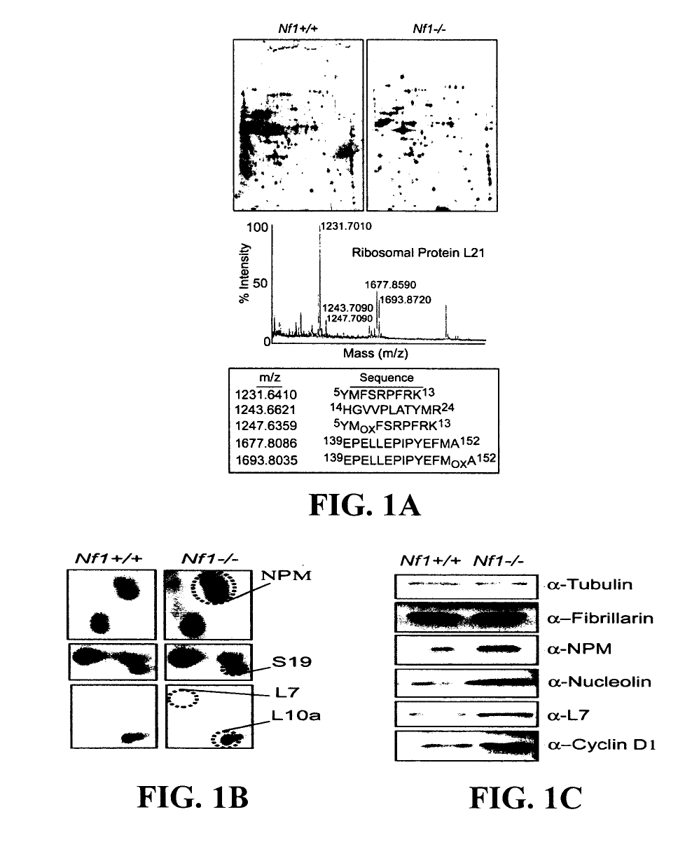

A large-scale proteomic analysis of primary astrocytes lacking neurofibromin was performed to determine whether loss of neurofibromin expression results in the activation of unique downstream signaling cascades. Proteins extracted from asynchronous wild-type (Nf1+/+) and Nf1−/− astrocytes were separated by two-dimensional electrophoresis using protein isoelectric points in the first dimension and protein molecular weights in the second dimension. When compared using PDQuest software, over 100 visualized proteins exhibited significant expression deviation (>1.5-fold) between Nf1+/+ and Nf1−/− extracts (FIG. 1A). Differentially expressed proteins were excised and analyzed by MALDI-TOF for initial identification. For example, ribosomal protein L21 was positively identified by peptide matching (FIG. 1A), and exhibited a 1.5-fold increase in protein expression in Nf1−/− astrocytes compared to wild-type astrocytes. While a number of differentially-expressed proteins remained unclassified in the proteomic databases (listed as ESTs or no protein matches), several other proteins involved in protein synthesis were positively identified. As shown in FIG. 1B, nucleophosmin (NPM), S19, L7, and L10a displayed increased expression in Nf1−/− astrocytes, and were subsequently positively identified by MALDI-TOF analysis.

The results from the proteomic analysis were confirmed by analyzing protein lysates from Nf1+/+ and Nf1−/− astrocytes by immunoblotting with antibodies recognizing initially-identified proteins from the mass spectrometry screen. In agreement with previous findings that Nf1−/− astrocytes proliferate at a greater rate than wild-type astrocytes, it was determined that cyclin D1 protein expression was increased in Nf1−/− astrocytes (FIG. 1C). Consistent with the proteomic analysis, NPM and L7 proteins were also significantly elevated in Nf1−/− astrocytes compared to wild-type astrocytes (FIG. 1C). NPM and L7 are nucleolar proteins involved in ribosomal RNA (rRNA) processing, assembly and nuclear export. Thus, it was investigated whether the expression of other nucleolar proteins involved in protein synthesis might be dysregulated in Nf1−/− astrocytes. Indeed, nucleolin, a nucleolar protein involved in ribosome biogenesis, was increased in Nf1−/− astrocytes (FIG. 1C). However, fibrillarin, a nucleolar protein responsible for early processing of 47S rRNA molecules, was unchanged in Nf1−/− astrocytes, suggesting that increases in nucleolar ribosome components following loss of neurofibromin expression were selective. Consistent with this notion, both NPM and nucleolin are positive regulators of cell growth, while other nucleolar ribosome components, such as fibrillarin and L11, either exhibit little influence on cell cycle progression or act as putative tumor suppressors. See Okuda M, et al., Nucleophosmin/B23 is a Target of CDK2/Cyclin E in Centrosome Duplication, CELL 103:127-140 (2000); Itahana K, et al., Tumor Suppressor ARF Degrades B23, a Nucleolar Protein Involved in Ribosome Biogenesis and Cell Proliferation, MOL. CELL 12:1151-1164 (2003); and Turck N, et al., Proteomic Analysis of Nuclear Proteins from Proliferative and Differentiated Human Colonic Intestinal Epithelial Cells, PROTEOMICS 4: 93-105 (2004). Notably, NPM is a potent oncogene whose continued expression is needed for efficient ribosome export and cell cycle progression, thus making it a downstream target for increased proliferation in Nf1−/− astrocytes. Additionally, the identification of numerous other ribosome-promoting factors in Nf1−/− astrocytes suggests that neurofibromin plays a more direct role in suppressing improper ribosome biogenesis and subsequent cell growth.

### Example 3

### Neurofibromin Loss Results in Increased Protein Synthesis in Astrocytes

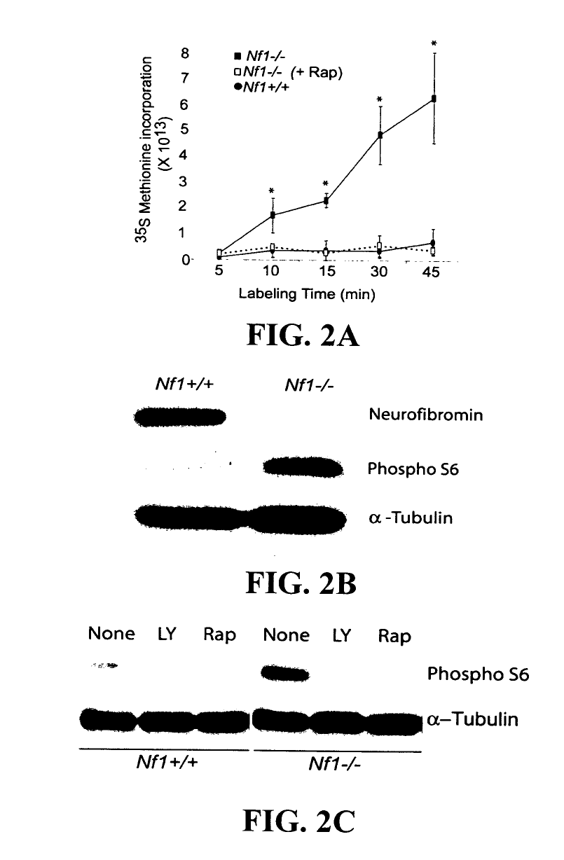

Translational regulation and control of ribosome biogenesis are vital cellular processes that directly impact on cell proliferation and growth. Activated RAS and AKT control protein translation, and the combination of activated RAS and AKT has been shown to recruit specific growth-promoting mRNAs to polysomes in glial cells. See Rajasekhar V K, et al., Oncogenic Ras and Akt Signaling Contribute to Glioblastoma Formation by Differential Recruitment of Existing mRNAs to Polysomes, MOL. CELL 12:889-901 (2003). Since several proteins associated with protein translation were aberrantly expressed in Nf1−/− astrocytes (see Example 2), an investigation was performed to determine whether the changes in protein expression were associated with an increase in the overall rate of protein synthesis. Nf1+/+ and Nf1−/− astrocytes were pulsed with ³⁵S-methionine, and the amount of radioactivity incorporated into newly synthesized proteins was determined. At all time points examined, significantly higher amounts of radioactivity were incorporated in Nf1−/− astrocytes compared to wild-type astrocytes (FIG. 2A). At the 45-min time point, there was an eight-fold increase in protein synthesis in Nf1−/− astrocytes relative to Nf1+/+ astrocytes. These results suggest that neurofibromin loss in astrocytes is associated with increased protein synthesis, likely reflecting increased protein translation.

Since neurofibromin functions as a negative regulator of RAS, the activation of a downstream RAS effector (ribosomal S6 protein) implicated in the regulation of mRNA translation was examined. See Montagne J, et al., Drosophila S6 Kinase: a Regulator of Cell Size, SCIENCE 285:2126-2129 (1999). Ribosomal S6 protein is activated by phosphorylation on specific residues (serine 235, 236, 240, and 244) primarily by its upstream kinase, ribosomal S6 kinase. See Radimerski T, et al. dS6K-Regulated Cell Growth is dPKB/dPI(3)K-Independent, but Requires dPDK1, NAT. CELL. BIOL. 4:251-255 (2002). To determine whether ribosomal S6 protein is hyperactivated in Nf1−/− astrocytes, the phosphorylation status of S6 in Nf1−/− and Nf1+/+ astrocytes was measured. As shown in FIG. 2B, loss of neurofibromin in astrocytes resulted in a 6-fold increase in S6 phosphorylation on serine residues 240 and 244.

One of the downstream targets of activated RAS is P13-Kinase. See Rodriguez-Viciana P, et al., Phosphatidylinositol-3-OH Kinase as a Direct Target of Ras, NATURE 370:527-532 (1994). In this regard, loss of neurofibromin expression is associated with activation of the PI3K-AKT signaling pathway in human NF1-associated pilocytic astrocytoma and in Nf1−/− mouse myeloid cells. See Lau N, et al., Loss of Neurofibromin is Associated with Activation of Ras/MAPK and PI3K/AKT Signalling in a Neurofibromatosis 1 Astrocytoma, J NEUROPATHOL. EXP. NEUROL. 59:759-767 (2000); and Donovan S, et al., Hyperactivation of Protein Kinase B and Erk Have Discrete Effects on Survival, Proliferation, and Cytokine Expression in Nf1-Deficient Myeloid Cells, CANCER CELL 2:507-514 (2002). Several studies have shown that activation of the PI3K-AKT leads to activation of the mammalian target of rapamycin (mTOR), which leads to phosphorylation of S6 kinase and activation of ribosomal S6. See Chung J, et al., PDGF- and Insulin-Dependent pp70S6k Activation Mediated by Phosphatidylinositol-3-OH Kinase, NATURE 370:71-75 (1994); and Gingras A C, et al., Regulation of Translation Initiation by FRAP/mTOR, GENES DEV. 15:807-26 (2001). Inhibition of S6 phosphorylation as a reflection of mTOR activity is achieved by blocking PI3K activation with the inhibitor LY294002, providing further evidence of PI3K-mediated mTOR activation. See Brunn G J, et al., Direct Inhibition of the Signaling Functions of the Mammalian Target of Rapamycin by the Phosphoinositide 3-Kinase Inhibitors, Wortmannin and LY294002, EMBO J. 15: 5256-67 (1996). To determine whether S6 hyperactivation in Nf1−/− astrocytes is downstream of PI3K and mTOR, S6 phosphorylation in both Nf1+/+ and Nf1−/− astrocytes in presence of a PI3K inhibitor (LY294002) and an mTOR inhibitor (rapamycin) was measured. As shown in FIG. 2C, S6 phosphorylation was completely inhibited in wild-type and Nf1−/− astrocytes at 50 μM LY 294002 and at 5 μM rapamycin, demonstrating the requirement for the PI3K/mTOR axis in promoting S6 activation in the absence of neurofibromin.

### Example 4

### Regulation of mTOR-Dependent S6 Activity in Astrocytes is Regulated by the Neurofibromin Gap-Related Domain

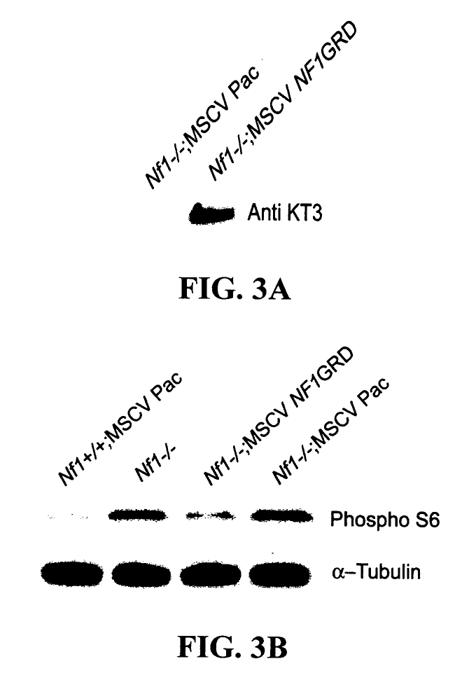

Previous studies have shown that neurofibromin growth regulation in multiple cell types requires residues within its RAS-GAP domain. See Hiatt K K, et al., J. BIOL. CHEM. 276:7240-7245 (2001). To determine whether the hyperactivation of S6 in Nf1−/− astrocytes reflects dysregulated RAS activity resulting from loss of neurofibromin RAS GAP function, the NF1GRD (GAP-related domain) was ectopically expressed in Nf1−/− astrocytes to restore neurofibromin GAP function. In Nf1−/− murine embryonic fibroblasts, expression of the NF1GRD was sufficient to restore normal growth. See Hiatt K K, et al., J. BIOL. CHEM. 276:7240-7245 (2001). To examine the effect of NF1GRD expression on S6 hyperactivation in Nf1−/− astrocytes, the NF1GRD was ectopically expressed by MSCV-mediated retroviral transduction (FIG. 3A). Transduction of Nf1−/− astrocytes with MSCV-NF1GRD, but not with MSCV-Pac (vector control), completely abrogated the hyperphosphorylation of S6 seen in Nf1−/− astrocytes (FIG. 3B), demonstrating that inactivation of RAS by neurofibromin is a key event in suppressing S6 hyperactivation and astrocyte proliferation. Transduction of wild-type astrocytes with either MSCV-NF1GRD or vector control virus did not affect S6 phosphorylation.

### Example 5

### Dysregulated K-RAS Activity Accounts for Hyperactivation of Ribosomal S6 in Nf1-Deficient Astrocytes

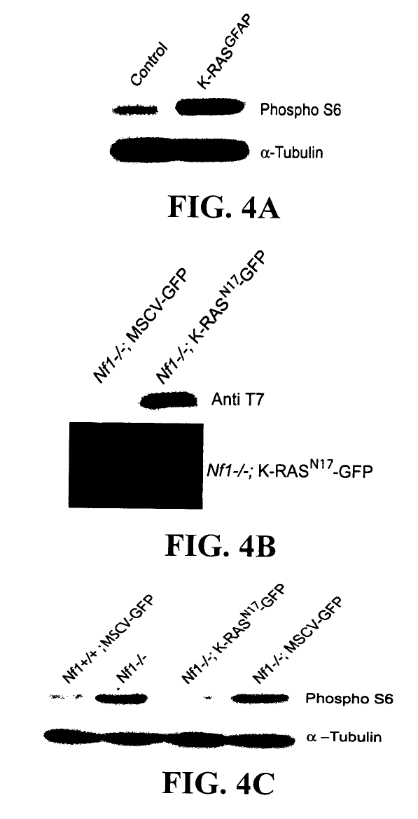

It has been previously shown that although all four RAS isoforms are expressed in astrocytes, the only RAS isoform activated as a result of Nf1 loss in astrocytes is K-RAS. See Dasgupta B, et al., Glioma Formation in Neurofibromatosis 1 Reflects Preferential Activation of K-RAS in Astrocytes, CANCER RES. 65:236-245 (2005). In this regard, activated K-RAS expression can substitute for Nf1 loss in astrocytes during optic nerve glioma formation in mice in vivo. To determine whether hyperactivated S6 in Nf1−/− astrocytes could also be mimicked by K-RAS activation in astrocytes, the phosphorylation status of S6 in K-RAS^{G12D} transgenic astrocytes was examined. In these experiments, a 9.6 fold increase in phosphorylated S6 in K-RAS^{G12D} astrocytes was observed compared to wild-type astrocytes (FIG. 4A).

It has been previously shown that the growth advantage of Nf1−/− astrocytes could be reversed by expressing dominant inhibitory K-RAS (K-RAS^{N17}) in Nf1−/− astrocytes. See Dasgupta B, et al., CANCER RES. 65:236-245 (2005). To determine whether the S6 hyperactivation resulted from dysregulated K-RAS activity in Nf1−/− astrocytes, K-RAS^{N17} was ectopically expressed in Nf1−/− astrocytes using MSCV-mediated retroviral transduction (FIG. 4B). As shown in FIG. 4C, dominant inhibitory K-RAS expression completely abrogated the increase in S6 phosphorylation observed in Nf1−/− astrocytes, whereas transduction of Nf1−/− astrocytes with MSCV-GFP (vector control), or Nf1+/+ astrocytes with either MSCV-GFP or MSCV-K-RAS^{N17} (not shown) did not affect S6 phosphorylation. These observations are consistent with previous reports demonstrating that activation of K-RAS can induce PI3K-dependent activation of the mTOR-S6 kinase pathway and result in transformation of various cell types. See Shao J, et al., Roles of Phosphatidylinositol 3′-Kinase and Mammalian Target of Rapamycin/p70 Ribosomal Protein S6 Kinase in K-Ras-Mediated Transformation of Intestinal Epithelial Cells, CANCER RES. 64:229-235 (2004). Collectively, the data support a model in which neurofibromin loss results in K-RAS-dependent and PI3K-regulated hyperactivation of the mTOR/S6K pathway. These hyperproliferative signals are relayed to the ribosome machinery, stimulating increases in protein synthesis, and ultimately culminating in uncontrolled cell growth.

### Example 6

### Genetically Engineered Nf1 Mouse Optic Pathway Glioma (OPG) and Human NF1-Associated Pilocytic Astrocytoma Exhibit Increased Ribosomal S6 Activation In Vivo

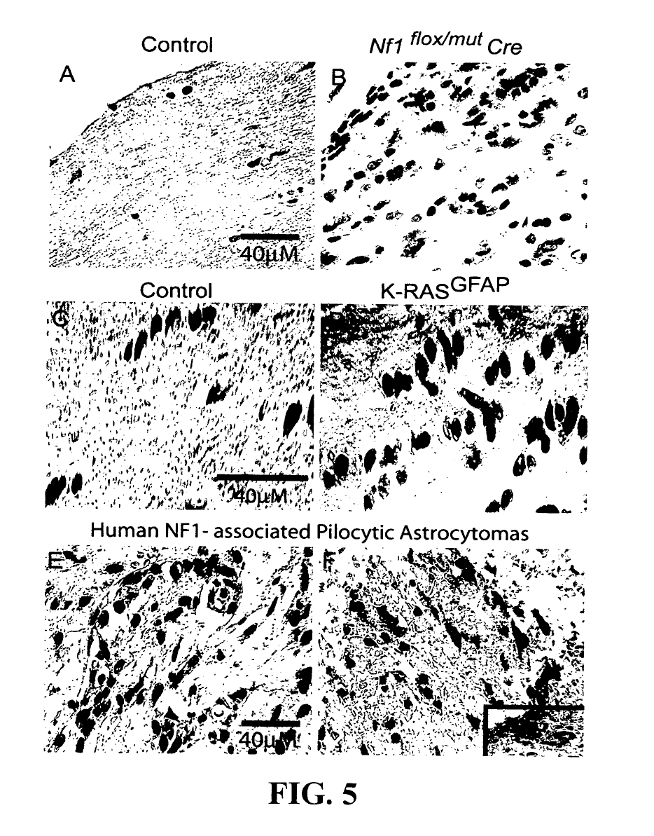

Since S6 was hyperactivated in Nf1−/− astrocytes in vitro, an investigation was performed to determine whether astrocytic tumors arising in genetically-engineered Nf1 murine glioma models exhibited S6 hyperactivation. For these experiments, immunohistochemistry on paraffin sections of tumors was performed using phospho-specific S6 antibodies. Previous studies from have shown that Nf1+/−mice either lacking Nf1 or expressing activated K-RAS in astrocytes develop low-grade optic nerve glioma (OPG). See Bajenaru M L, et al., Optic Nerve Glioma in Mice Requires Astrocyte Nf1 Gene Inactivation and Nf1 Brain Heterozygosity, CANCER RES. 63:8573-8577 (2003); and Dasgupta B, et al., CANCER RES. 65:236-245 (2005). Analysis of optic nerve tumors from GFAPCre; Nf1^{flox/mut} mice showed phosphorylated S6 expression in neoplastic astrocytes within the hypercellular optic chiasm (FIG. 5B). No phosphorylated S6 was observed in the optic chiasm of control mice (FIG. 5A). Similarly, in the Nf1+/−; K-RAS^{GFAP} mouse OPG model, the neoplastic astrocytes with atypical nuclei in the optic chiasm also expressed phosphorylated S6 (FIG. 5D). No expression of phosphorylated S6 was observed in the optic chiasm of control mice (FIG. 5C).

Next, an investigation was performed to determine whether S6 hyperactivation was also observed in human NF1-associated pilocytic astrocytoma. Four out of six NF1-associated pilocytic astrocytomas showed expression of phosphorylated S6 (FIG. 5E, F). In one of these NF1-associated WHO grade I astrocytomas, phosphorylated S6 expression was observed in foci of hypercellular tumoral regions containing atypical nuclei (FIG. 5F, inset). Collectively, these results demonstrate that neurofibromin loss leads to hyperactivation of the mTOR/S6 pathway in astrocytes in vitro and in NF1-associated mouse and human astrocytomas in vivo, underscoring the physiological relevance of neurofibromin-regulated S6 hyperactivation in NF1-associated tumor formation.

### Example 7

### Rapamycin Inhibition of Ribosomal S6 Abrogates the Hyperproliferation Observed in Nf1−/− Astrocytes

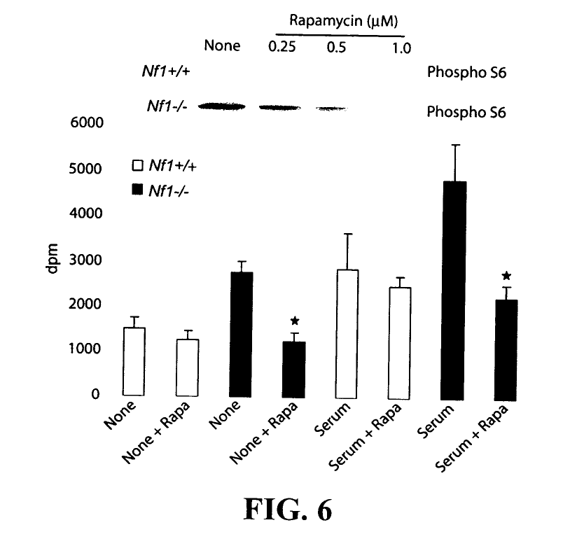

Previous studies have shown that Nf1−/− astrocytes exhibit a 2-3 fold increase in proliferation compared to wild-type astrocytes. See Bajenaru M L, et al., Astrocyte-Specific Inactivation of the Neurofibromatosis 1 Gene (NF1) is Insufficient for Astrocytoma Formation, MOL. CELL BIOL. 22:5100-5113 (2002). To determine whether the growth advantage of Nf1-deficient astrocytes reflected increased mTOR signaling, Nf1−/− astrocyte proliferation after rapamycin treatment was measured. First, the minimal dose of rapamycin which could reduce Nf1−/− astrocyte S6 phosphorylation to levels seen in Nf1+/+ astrocytes without causing significant cell death was determined. As shown in FIG. 6 (inset), 1 μM rapamycin significantly inhibited S6 phosphorylation in Nf1−/− astrocytes, but had no effect on S6 phosphorylation in wild-type astrocytes. Using Trypan blue exclusion, greater than 98% cell survival at this dose of rapamycin was observed. Using this minimal dose (1 μM) of rapamycin, it was found that rapamycin significantly inhibited the proliferation of Nf1−/− astrocytes (55±9.5% reduction), with minimal effects on wild-type astrocyte basal proliferation (16±4%). Similar reduction in proliferation of Nf1−/− astrocytes in presence of serum was also observed (FIG. 6). These results demonstrate that the increased proliferation observed in Nf1−/− astrocytes can be inhibited by blocking hyperactivation of the mTOR/S6 pathway without deleterious effects on normal cells. In this regard, Nf1−/− astrocytes appear to be dependent on continued mTOR signaling and as such, are much more susceptible to rapamycin treatment. Similar instances of drug sensitivity have recently been observed in cells lacking PTEN or in tumors overexpressing specific activating EGFR mutants. See Grunwald V, et al., Inhibitors of mTOR Reverse Doxorubicin Resistance Conferred by PTEN Status in Prostate Cancer Cells, CANCER RES. 62:6141-5 (2002); and Albanell J, et al., Activated Extracellular Signal-Regulated Kinases: Association with Epidermal Growth Factor Receptor/Transforming Growth Factor Alpha Expression in Head and Neck Squamous Carcinoma and Inhibition by Anti-Epidermal Growth Factor Receptor Treatments, CANCER RES. 61:6500-6510 (2001). In these cases, selection of cells expressing EGFR mutants or cells lacking PTEN conferred dependence on these hyperactivated pathways for continued proliferation, such that inhibition of these pathways with selective inhibitors resulted in dramatically attenuated cell proliferation. In astrocytes lacking neurofibromin, there is hyperactivation of the mTOR/S6 pathway, which may serve as the predominant signal upon which Nf1-deficient astrocytes depend for continued proliferation.

The above detailed description is intended only to acquaint others skilled in the art with the invention, its principles, and its practical application so that others skilled in the art may adapt and apply the invention in its numerous forms, as they may be best suited to the requirements of a particular use. This invention, therefore, is not limited to the above embodiments, and may be variously modified.

All references cited above are incorporated by reference into this patent. The discussion of those references is intended merely to summarize the assertions made by their authors. No admission is made that any reference (or a portion of any reference) is relevant prior art. Applicants reserve the right to challenge the accuracy and pertinence of the cited references.

The words “comprise”, “comprises”, and “comprising” are to be interpreted inclusively rather than exclusively.

We claim:

1. A method for treating neurofibromatosis 1 in an animal, wherein the method comprises administering to the animal an amount of rapamycin or a pharmaceutically-acceptable salt thereof.

2. The method of claim 1, wherein the animal is a human.

3. The method of claim 1, wherein the amount of rapamycin or a salt thereof that is administered to the animal is a therapeutically-effective amount.

4. The method of claim 1, wherein the method further comprises administering to the animal an amount of an anti-tumor compound or a pharmaceutically-acceptable salt thereof.

5. The method of claim 4, wherein the amount of rapamycin (or a salt thereof) and the amount of anti-tumor compound (or a salt thereof) together comprise a therapeutically-effective amount.

6. The method of claim 1, wherein the animal has a benign tumor.

7. The method of claim 6, wherein the tumor comprises a neurofibroma.

8. The method of claim 6, wherein the tumor comprises an astrocytoma.

9. The method of claim 1, wherein the animal has a skeletal abnormality.

10. The method of claim 1, wherein the animal has a learning disability.

11. A method for treating neurofibromatosis 1 in an animal, wherein the method comprises administering to the animal an amount of a rapamycin analog, a rapamycin prodrug, or a pharmaceutically-acceptable salt of the analog or prodrug.

12. The method of claim 11, wherein the rapamycin analog administered is selected from the group consisting of:

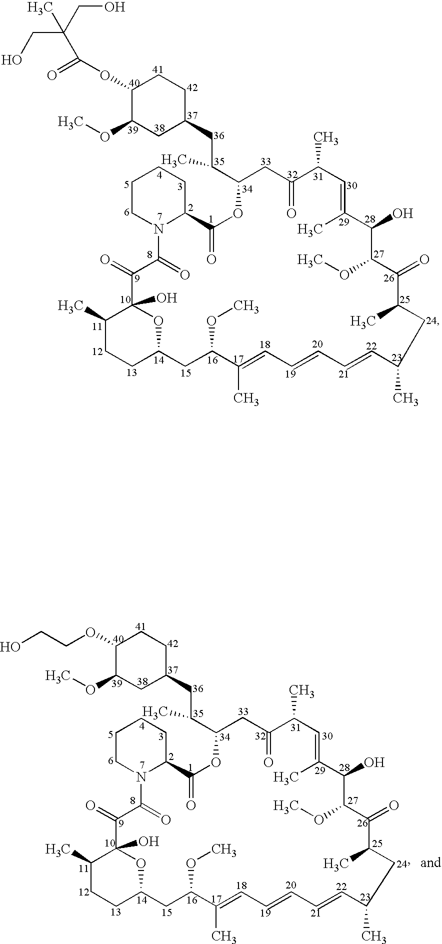

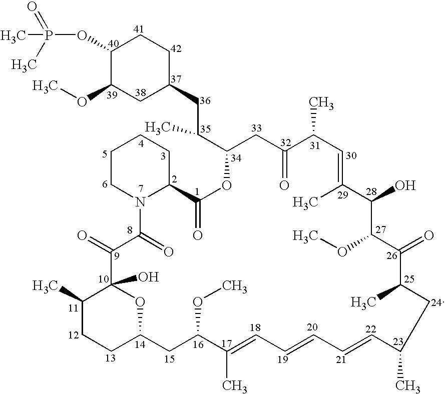

13. The method of claim 11, wherein the amount of rapamycin analog, rapamycin prodrug, or a salt of the analog or prodrug that is administered to the animal is a therapeutically-effective amount.

14. The method of claim 11, wherein the method further comprises administering to the animal an amount of an anti-tumor compound or a pharmaceutically-acceptable salt thereof.

15. The method of claim 14, wherein the amount of rapamycin analog or prodrug (or a salt thereof) and the amount of anti-tumor compound (or a salt thereof) together comprise a therapeutically-effective amount.

16. The method of claim 11, wherein the animal has a benign tumor.

17. The method of claim 16, wherein the tumor is selected from the group consisting of a neurofibroma and an astrocytoma.

18. The method of claim 11, wherein the animal has a skeletal abnormality.

19. The method of claim 11, wherein the animal has a learning disability.

20. A method for determining if an animal neurofibromatosis 1-associated tumor is likely to respond to treatment with rapamycin, a rapamycin analog, a rapamycin prodrug, or a salt of rapamycin or the analog or prodrug, wherein the method comprises examining the level of phosphorylation of ribosomal S6 protein in a sample obtained from the tumor.

21. The method of claim 20, wherein the tumor is selected from the group consisting of a neurofibroma, an astrocytoma, and a malignant peripheral sheath tumor.

22. The method of claim 20, wherein the sample obtained from the tumor is prepared as a paraffin-embedded biopsy.

23. The method of claim 20, wherein examining the level of phosphorylation of ribosomal S6 protein comprises treating the sample with an antibody that recognizes a phosphorylated serine residue of ribosomal S6 protein.

24. A kit for treating neurofibromatosis 1 in an animal, wherein the kit comprises a dosage form comprising an amount of rapamycin, a rapamycin analog, a rapamycin prodrug, or a pharmaceutically-acceptable salt of rapamycin or the analog or prodrug.

25. The kit of claim 24, wherein the amount of rapamycin, rapamycin analog, rapamycin prodrug, or pharmaceutically-acceptable salt of rapamycin or the analog or prodrug is a therapeutically-effective amount.

26. The kit of claim 24, wherein the kit further comprises a dosage form comprising an amount of anti-tumor compound or a pharmaceutically-acceptable salt thereof.

27. The kit of claim 26, wherein the amount of rapamycin or rapamycin analog or prodrug (or a salt thereof) and the amount of anti-tumor compound (or a salt thereof) together comprise a therapeutically-effective amount.
